(19) United States
(12) Patent Application Publication
(10) Pub. No.: US 2011/0190393 A1
(43) Pub. Date: Aug. 4, 2011

(54) NOVEL AND EFFICIENT METHOD FOR THE SYNTHESIS OF AN AMINO ACID

(75) Inventors: Vinayak Gore, Maharashtra (IN); Maheshkumar Gadakar, Maharashtra (IN); Dattatreya Shinde, Maharashtra (IN)

(73) Assignee: Generics (UK) Limited, Hartfordshire (GB)

(21) Appl. No.: 12/992,489

(22) PCT Filed: Jun. 3, 2009
(86) PCT No.: PCT/GB09/50612

§ 371 Date: Apr. 8, 2011

(30) Foreign Application Priority Data
Jun. 3, 2008 (IN) 985/KOL/2008

(51) Int. Cl. A61K 31/225; C07C 205/49; A61K 31/195; C07C 229/06; A61P 25/08; A61P 25/00

(52) U.S. Cl. 514/547; 562/553; 514/561; 560/171

(57) ABSTRACT

The present invention relates to a novel process for the preparation of γ-amino acids, such as (±)-3-(aminomethyl)-5-methyl-hexanoic acid (1), which is a key intermediate in the preparation of the potent anticonvulsant pregabalin, (S)-(+)-3-(aminomethyl)-5-methyl-hexanoic acid (2), and its analogues.

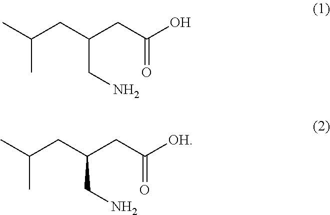

### CROSS-REFERENCE TO RELATED APPLICATION(S)

This application is a Section 371 National Stage Application of International No. PCT/GB2009/050612, filed 3 Jun. 2009 and published as WO 2009/147434 A1 on 10 Dec. 2009, which claims priority from the IN Patent Application No. 985/KOL/2008, filed 3 Jun. 2008, the contents of which are incorporated herein in their entirety for all purposes.

### FIELD OF THE INVENTION

The present invention relates to a novel process for the preparation of γ-amino acids, such as (±)-3-(aminomethyl)-5-methyl-hexanoic acid (1), which is a key intermediate in the preparation of the potent anticonvulsant pregabalin, (S)-(+)-3-(aminomethyl)-5-methyl-hexanoic acid (2), and its analogues.

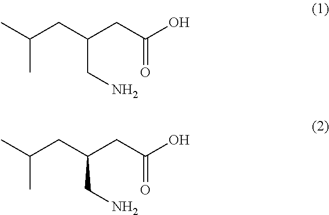

### BACKGROUND OF THE INVENTION

(±)-3-(Aminomethyl)-5-methyl-hexanoic acid, or (±)-β-isobutyl-γ-amino-butyric acid, or (±)-isobutyl-GABA, hereafter called racemic pregabalin (1), was first reported in Synthesis, 1989, 953. The synthetic process reported involved the addition of nitromethane to an ethyl 2-alkenoate and the nitro ester thus formed was reduced using palladium on carbon. Subsequent hydrolysis using hydrochloric acid afforded racemic pregabalin (1) as the hydrochloride salt. The free base of racemic pregabalin (1) was then prepared by ion exchange chromatography.

An alternative process reported in U.S. Pat. No. 5,637,767 describes the condensation of isovaleraldehyde with diethyl malonate. The 2-carboxy-2-alkenoic acid thus formed was reacted with a cyanide source, specifically potassium cyanide. The cyano diester product was decarboxylated by heating with sodium chloride in DMSO and water and hydrolyzed using KOH to give the potassium salt of a cyano acid. This was hydrogenated in situ using sponge nickel and neutralized with acetic acid to give racemic pregabalin (1).

A further process for preparing racemic pregabalin hydrochloride has been reported in US 2005/0043565. This process involved a Wittig-Horner reaction between isovaleraldehyde and triethyl phosphonoacetate to give the ethyl 2-alkenoate. Addition of nitromethane using TBAF, followed by hydrogenation using Raney nickel afforded the lactam, which was hydrolyzed using HCl to form the hydrochloride salt of the amino acid.

The major disadvantage of the process disclosed in the Synthesis 1989 article is the use of an expensive lithium bis(trimethylsilylamide) reagent at very low temperature conditions (−78° C.) which is difficult to conduct on large scale.

The process reported in U.S. Pat. No. 5,637,767 suffers from various disadvantages, which make it difficult to achieve an acceptable impurity profile as per ICH guidelines and an acceptable chiral purity of pregabalin (2). The process reported in U.S. Pat. No. 5,637,767 uses highly toxic KCN, which should be avoided, and the use of sponge nickel is potentially hazardous. In addition the process reported in U.S. Pat. No. 5,637,767 involves lengthy reaction times and very high temperatures, which leads to the formation of degradation products and low yields.

The process reported in US 2005/0043565 involves the use of phosphorous compounds, which are very difficult to eliminate from the final product, lengthy reaction times and high-pressure reactions. In addition the process affords the hydrochloride salt instead of the free base and it is well known that there are practical difficulties in the isolation of amino acids from aqueous media due to the formation of zwitterionic species. The formation of the HCl salt of racemic pregabalin (1) necessitates an aqueous work-up, which leads to poor yields and lengthy work-up procedures.

### DEFINITIONS

For the purposes of the present invention, an “alkyl” group is defined as a monovalent saturated hydrocarbon, which may be straight-chained or branched, or be or include cyclic groups. An alkyl group may optionally be substituted, and may optionally include one or more heteroatoms N, O or S in its carbon skeleton. Preferably an alkyl group is straight-chained or branched. Preferably an alkyl group is not substituted. Preferably an alkyl group does not include any heteroatoms in its carbon skeleton. Examples of alkyl groups are methyl, ethyl, n-propyl, i-propyl, n-butyl, i-butyl, tert-butyl, n-pentyl, cyclopentyl, cyclohexyl and cycloheptyl groups. Preferably an alkyl group is a C₁₋₁₂ alkyl group (i.e. an alkyl group containing from 1 to 12 carbon atoms), preferably a C₁₋₆ alkyl group. Preferably a cyclic alkyl group is a C₃₋₁₂ cyclic alkyl group, preferably a C₅₋₇ cyclic alkyl group. An “alkylene” group is similarly defined as a divalent alkyl group.

An “alkenyl” group is defined as a monovalent hydrocarbon, which comprises at least one carbon-carbon double bond, which may be straight-chained or branched, or be or include cyclic groups. An alkenyl group may optionally be substituted, and may optionally include one or more heteroatoms N, O or S in its carbon skeleton. Preferably an alkenyl group is straight-chained or branched. Preferably an alkenyl group is not substituted. Preferably an alkenyl group does not include any heteroatoms in its carbon skeleton. Examples of alkenyl groups are vinyl, allyl, but-1-enyl, but-2-enyl, cyclohexenyl and cycloheptenyl groups. Preferably an alkenyl group is a C₂₋₁₂ alkenyl group, preferably a C₂₋₆ alkenyl group. Preferably a cyclic alkenyl group is a C₃₋₁₂ cyclic alkenyl group, preferably a C₅₋₇ cyclic alkenyl group. An “alkenylene” group is similarly defined as a divalent alkenyl group.

An “alkynyl” group is defined as a monovalent hydrocarbon, which comprises at least one carbon-carbon triple bond, which may be straight-chained or branched, or be or include cyclic groups. An alkynyl group may optionally be substituted, and may optionally include one or more heteroatoms N, O or S in its carbon skeleton. Preferably an alkynyl group is straight-chained or branched. Preferably an alkynyl group is not substituted. Preferably an alkynyl group does not include any heteroatoms in its carbon skeleton. Examples of alkynyl groups are ethynyl, propargyl, but-1-ynyl and but-2-ynyl groups. Preferably an alkynyl group is a C₂₋₁₂ alkynyl group, preferably a C₂₋₆ alkynyl group. Preferably a cyclic alkynyl group is a C₃₋₁₂ cyclic alkynyl group, preferably a C₅₋₇ cyclic alkynyl group. An “alkynylene” group is similarly defined as a divalent alkynyl group.

An “aryl” group is defined as a monovalent aromatic hydrocarbon. An aryl group may optionally be substituted, and may optionally include one or more heteroatoms N, O or S in its carbon skeleton. Preferably an aryl group is not substituted. Preferably an aryl group does not include any heteroatoms in its carbon skeleton. Examples of aryl groups are phenyl, naphthyl, anthracenyl and phenanthrenyl groups. Preferably an aryl group is a C₄₋₁₄ aryl group, preferably a C₆₋₁₀ aryl group. An “arylene” group is similarly defined as a divalent aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

For the purposes of this invention, an optionally substituted alkyl, alkenyl, alkynyl, aryl, arylalkyl, arylalkenyl, arylalkynyl, alkylaryl, alkenylaryl or alkynylaryl group may be substituted with one or more of —F, —Cl, —Br, —I, —CF₃, —CCl₃, —CBr₃, —Cl₃, —OH, —SH, —NH₂, —CN, —NO₂, —COOH, —R^{α}—O—R^{β}, —R^{α}—S—R^{β}, —R^{α}—SO—R^{β}, —R^{α}—SO₂—R^{β}, —R^{α}—SO₂—OR^{β}, —R^{α}O—SO₂—R^{β}, —R^{α}—SO₂—N(R^{β})₂, —R^{α}—NR^{β}—SO₂—R^{β}, —R^{α}O—SO₂—OR^{β}, —R^{α}O—SO₂—N(R^{β})₂, —R^{α}—NR^{β}—SO₂—OR^{β}, —R^{α}—NR^{β}—SO₂—N(R^{β})₂, —R^{α}—N(R^{β})₂, —R^{α}—N(R^{β})₃⁺, —R^{α}—P(R^{β})₂, —R^{α}—Si(R^{β})₃, —R^{α}—CO—R^{β}, —R^{α}—CO—OR^{β}, —R^{α}O—CO—R^{β}, —R^{α}—CO—N(R^{β})₂, —R^{α}—NR^{β}—CO—R^{β}, —R^{α}O—CO—OR^{β}, —R^{α}O—CO—N(R^{β})₂, —R^{α}—NR^{β}—CO—OR^{β}, —R^{α}—NR^{β}—CO—N(R^{β})₂, —R^{α}—CS—R^{β}, —R^{α}—CS—OR^{β}, —R^{α}O—CS—R^{β}, —R^{α}—CS—N(R^{β})₂, —R^{α}—NR^{β}—CS—R^{β}, —R^{α}O—CS—OR^{β}, —R^{α}O—CS—N(R^{β})₂, —R^{α}—NR^{β}—CS—OR^{β}, —R^{α}—NR^{β}—CS—N(R^{β})₂, —R^{β}, a bridging substituent such as —O—, —S—, —NR^{β}— or —R^{α}—, or a π-bonded substituent such as ═O, ═S or ═NR^{β}. In this context, —R^{α}— is independently a chemical bond, or a C₁-C₁₀ alkylene, C₂-C₁₀ alkenylene or C₂-C₁₀ alkynylene group. —R^{β} is independently hydrogen, or an unsubstituted C₁-C₆ alkyl or unsubstituted C₆-C₁₀ aryl group. Optional substituent(s) are preferably taken into account when calculating the total number of carbon atoms in the parent group substituted with the optional substituent(s). Preferably an optionally substituted alkyl, alkenyl, alkynyl, aryl, arylalkyl, arylalkenyl, arylalkynyl, alkylaryl, alkenylaryl or alkynylaryl group is not substituted with a bridging substituent. Preferably an optionally substituted alkyl, alkenyl, alkynyl, aryl, arylalkyl, arylalkenyl, arylalkynyl, alkylaryl, alkenylaryl or alkynylaryl group is not substituted with a π-bonded substituent. Preferably a substituted group comprises 1, 2 or 3 substituents, more preferably 1 or 2 substituents, and even more preferably 1 substituent.

Preferably an optionally substituted alkyl, alkenyl, alkynyl, aryl, arylalkyl, arylalkenyl, arylalkynyl, alkylaryl, alkenylaryl or alkynylaryl group is substituted with one or more halo, alkylhalo, hydroxy, thio, nitro, amino, alkyl, alkoxy or carboxy groups.

Any optional substituent may be protected. Suitable protecting groups for protecting optional substituents are known in the art, for example, from “Protective Groups in Organic Synthesis” by T. W. Greene and P. G. M. Wuts (Wiley-Interscience, 3^{rd} edition, 1999 and 4^{th} edition, 2006).

An “alkoxy” group is defined as a —O-alkyl, —O-alkenyl, —O-alkynyl, —O-aryl, —O-arylalkyl, —O-arylalkenyl, —O-arylalkynyl, —O-alkylaryl, —O-alkenylaryl or —O-alkynylaryl group. Preferably an “alkoxy” group is a —O-alkyl or —O-aryl group. More preferably an “alkoxy” group is a —O-alkyl group. An “alkoxide” is similarly defined as an alkoxy group with a negative charge on the oxygen atom in place of the connecting chemical bond.

A “halo” group is a fluoro, chloro, bromo or iodo group.

An “alkylhalo” group is an alkyl group substituted with one or more halo groups.

A “hydroxy” group is a —OH group. A “thio” group is a —SH group. A “nitro” group is a —NO₂ group. An “amino” group is a —NH₂ group. A “carboxy” group is a —CO₂H group.

The compounds of the present invention, including any of the starting materials, intermediates or products of the processes of the present invention, can be used either in their free acid- or base-form, or as a salt such as an acid addition salt or one formed between a carboxylic acid functionality and a suitable cation. Preferably where a salt is used, the salt is a pharmaceutically acceptable salt.

Acid addition salts are preferably non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono- or di-acid addition salt. A preferred salt is a hydrohalogenic, sulfuric, phosphoric or organic acid addition salt. A more preferred salt is a hydrochloric acid addition salt.

In addition to pharmaceutically acceptable acid addition salts, other acid addition salts are included in the present invention, since they have the potential to serve as intermediates in the purification or preparation of other, for example, pharmaceutically acceptable, acid addition salts, or are useful for identification, characterisation, preparation or purification of the free base.

Suitable cations for forming a salt with a carboxylic acid functionality of a compound of the present invention include, but are not limited to lithium, sodium, potassium, magnesium, calcium and ammonium. The salt may be a mono-, di- or tri-salt. Preferably the salt is a mono- or di-lithium, sodium, potassium, magnesium, calcium or ammonium salt. More preferably the salt is a mono- or di-sodium salt.

It is preferred however that the starting materials, intermediates and products of the processes of the present invention are used in their free acid- or base-form except where stated otherwise.

The γ-amino acids of the present invention may have at least one chiral centre and therefore can exist in at least two stereoisomeric forms. For the purposes of the present invention, a γ-amino acid with one chiral centre is “racemic” if it comprises the two stereoisomers in a ratio of from 60:40 to 40:60, preferably in a ratio of about 50:50. A amino acid is “enantiomerically enriched”, if it comprises 60% or more of only one stereoisomer, preferably 70% or more, preferably 80% or more, preferably 90% or more. A γ-amino acid is “enantiomerically pure”, if it comprises 95% or more of only one stereoisomer, preferably 98% or more, preferably 99% or more, preferably 99.5% or more, preferably 99.9% or more.

For the purposes of the present invention, a γ-amino acid is “substantially free” of lactam impurity (3a), such as lactam impurity (3b), if it comprises less than 3% lactam impurity, preferably less than 2%, preferably less than 1%, preferably less than 0.5%, preferably less than 0.1%. The “lactam impurity” is the lactam (3a), such as racemic lactam (3b), or an enantiomer thereof, obtained by an intra-molecular condensation reaction of the respective γ-amino acid such as racemic pregabalin or pregabalin, wherein R′ and R″ are as defined below.

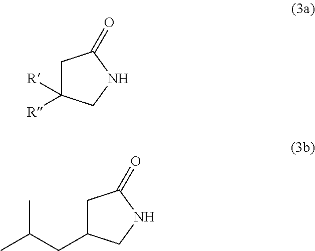

### SUMMARY OF THE INVENTION

The difficulties encountered in the prior art when preparing racemic pregabalin (1) have been successfully overcome in the present invention. The process of the present invention when applied to the synthesis of pregabalin uses isovaleraldehyde as a key starting material to synthesize racemic pregabalin (1). The racemic pregabalin (1) prepared by the present invention can be subsequently resolved to afford optically pure pregabalin (2). Alternatively, instead of resolving racemic pregabalin (1), any of the process intermediates can be resolved. The resolution can be done by following well-established and reported routes. For example, U.S. Pat. No. 5,637,767, which is herein incorporated by reference in its entirety, reports the resolution of racemic pregabalin (1) to pregabalin (2) by selective crystallisation with (S)- or (R)-mandelic acid.

The present invention provides a process for the preparation of racemic pregabalin (1) and other γ-amino acids by a high yielding, convenient and short route, which also avoids the use of hazardous and/or environmentally unsuitable reagents.

The present invention provides an efficient, simple and non-hazardous process for the preparation of γ-amino acids, such as racemic pregabalin (1), pregabalin (2) and their analogues.

The present invention also provides a commercially acceptable process with simple and precise experimental parameters to afford very pure racemic pregabalin (1), which will easily provide pregabalin (2) meeting the requirements of ICH guidelines.

Therefore, a first aspect of the present invention provides a process for the preparation of a γ-amino acid VI, comprising one or more steps selected from:

(i) the reaction of carbonyl compound I with nitromethane to form alcohol II:

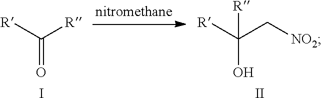

and/or
(ii) the conversion of alcohol II to intermediate IV:

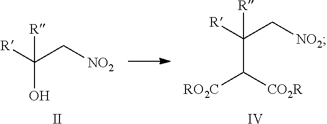

and/or
(iii) the conversion of intermediate IV to γ-nitro acid V, followed by the reduction of γ-nitro acid V to γ-amino acid VI:

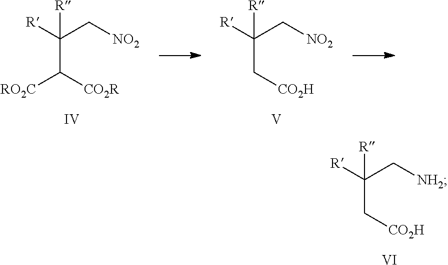

wherein each R is independently an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
wherein R′ and R″ are independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, or both R′ and R″ together with the carbon atom to which they are attached form a cyclic alkyl or cyclic alkenyl group, each of which may optionally be substituted, and each of which may optionally include one or more heteroatoms N, O or S in its carbon skeleton.

For the avoidance of doubt it should be noted that where a process of the invention is for the preparation of a compound and comprises a step, it is to be understood that said step is an integral part of the process, such that the end product of the step is ultimately converted into the desired compound.

In one embodiment of the first aspect of the present invention, the process comprises two of steps (i) to (iii), such as steps (i) and (ii), or steps (i) and (iii), or steps (ii) and (iii). Alternatively or in addition, the process may comprise step (ii) and the conversion of intermediate IV to γ-nitro acid V, as set out in step (iii) above. Preferably the process comprises all three of steps (i) to (iii).

In another embodiment of the first aspect of the present invention, each R contains from 1 to 12 carbon atoms, or from 1 to 6 carbon atoms. Optionally each R is the same. Preferably each R is independently an alkyl group such as a methyl, ethyl, propyl or butyl group. Most preferably each R is a methyl group.

In any embodiment of the first aspect of the present invention, it is preferred that the atoms by which both R′ and R″ are attached to the carbonyl group are either hydrogen or carbon. Similarly it is preferred that the atoms by which both R groups are connected to the oxygen of the carboxylic groups are not heteroatoms.

In one embodiment of the first aspect of the present invention, R′ and R″ are independently hydrogen or contain from 1 to 12 carbon atoms, or from 1 to 6 carbon atoms. In one preferred embodiment, one of R′ and R″ is hydrogen, optionally wherein the other is not hydrogen.

In another embodiment of the first aspect of the present invention, R′ and R″ are independently hydrogen or an alkyl group, preferably a C₁₋₆ alkyl group, or both R′ and R″ together with the carbon atom to which they are attached form a cyclic alkyl group, preferably a C₅₋₇ cyclic alkyl group. In one preferred embodiment, one of R′ and R″ is hydrogen and the other is i-butyl. In another preferred embodiment, both R′ and R″ together with the carbon atom to which they are attached form a cyclohexyl group.

In yet another embodiment of the first aspect of the present invention, a carbanion of nitromethane is generated in step (i) with a base. Optionally the base is not a primary or secondary amine, and preferably is not an amine. Preferably the base is a hydride, an alkoxide or a hydroxide, such as an alkali metal hydride, alkoxide or hydroxide. More preferably the base is an alkoxide. Exemplary alkoxides include for instance MeO⁻, EtO⁻, i-PrO⁻, t-BuO⁻ and PhO⁻. A preferred alkoxide is methoxide, most preferably sodium methoxide.

Where a base is used, it is preferably used in a catalytic amount such as 0.001 to 0.040 molar equivalents (eq), more preferably about 0.015 molar equivalents. The preferred quantity of nitromethane with respect to carbonyl compound I is 1 to 6 molar equivalents, more preferably about 2 molar equivalents.

Step (i) is optionally carried out in an aprotic solvent, preferably an ether solvent or a dipolar aprotic solvent such as N,N-dimethylformamide, dimethyl sulfoxide or acetonitrile. Preferably step (i) is carried out in an ether solvent such as tetrahydrofuran, diisopropyl ether, tert-butyl methyl ether, diethyl ether, or mixtures thereof. Most preferably step (i) is carried out in tetrahydrofuran.

In one embodiment of the first aspect of the present invention, the conversion of step (ii) comprises the substitution of the hydroxyl group of alcohol II to give intermediate IIIa:

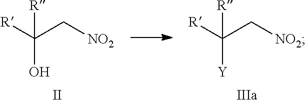

wherein Y is a suitable leaving group.

Intermediate IIIa may be generated for instance from intermediate II via an S_{N}2 displacement of an activated hydroxyl group by Y⁻. Preferably the activated hydroxyl group is generated in-situ.

Y may be for instance a halo group such as —Cl, —Br or —I. Preferably Y is —Br. Preferably when Y is a halo group, intermediate IIIa is generated from intermediate II using Y₂ and R^{x}₃P, or using HY, PY₃, PY₅, an N-halosuccinimide or SOY₂, wherein each R^{x} is independently selected from an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton. Preferably R^{x}₃P is triphenylphosphine. Alternatively when Y is a halo group, intermediate IIIa may be generated from intermediate II using an azodicarboxylate (such as diethyl azodicarboxylate), an alkyl halide (such as methyl iodide) and R^{x}₃P (such as triphenylphosphine), wherein R^{x} is as defined above.

In another embodiment of the first aspect of the present invention, the conversion of step (ii) comprises the activation of the hydroxyl group of alcohol II to give intermediate IIIb:

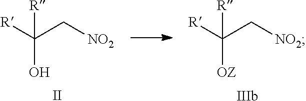

wherein Z is any group capable of enhancing the capacity of a hydroxyl group as a leaving group.

Z may be for instance selected from a —SO₂R^{a}, —SO₂OR^{a}, —NO₂, —COR^{a}, —P(═O)(OR^{a})₂ or —B(OR^{α})₂ group, wherein each R^{a} is independently selected from hydrogen, a halogen, or an optionally substituted alkyl, alkenyl, alkynyl, aryl, arylalkyl, arylalkenyl or arylalkynyl group, and wherein any two R^{a} groups may together with the atoms to which they are attached form a ring. Preferably each R^{a} is independently selected from an alkyl, aryl or arylalkyl group optionally substituted with one or more groups selected from —F, —Cl, —Br or —NO₂.

In one embodiment, Z is selected from a —SO₂R^{a}, —SO₂OR^{a} or —COR^{a} group. For instance, Z may be selected from a tosylate, brosylate, nosylate, mesylate, tresylate, nonaflate or triflate group. Alternatively, Z may be a —COR^{a} group, in which case R^{a} is preferably a C₁₋₁₂ alkyl, aryl or arylalkyl group optionally substituted with one or more groups selected from —F, —Cl, —Br or —NO₂, and more preferably R^{a} is a C₁₋₆ alkyl group optionally substituted with one or more groups selected from —F, —Cl or —Br. Most preferably Z is an acetyl or trifluoroacetyl group.

Where Z is a —COR^{a} group, it may be generated for instance by the reaction of the hydroxyl group of alcohol II with an acid chloride such as ClCOR^{a}, or an acid anhydride such as R^{a} C(O)OC(O)R^{a}. Preferably acetic anhydride or trifluoroacetic anhydride is used. The acid chloride or acid anhydride may be used for instance in an amount of from 1 to 6 molar equivalents relative to the alcohol II, preferably in an amount of from 1 to 2 molar equivalents, more preferably about 1.3 molar equivalents.

The generation of intermediate IIIa or of intermediate IIIb in step (ii) is optionally carried out in an aprotic solvent, preferably an ether solvent or a dipolar aprotic solvent such as N,N-dimethylformamide, dimethyl sulfoxide or acetonitrile. Preferably the generation of intermediate IIIa or IIIb is carried out in an ether solvent such as tetrahydrofuran, diisopropyl ether, tert-butyl methyl ether, diethyl ether, or mixtures thereof. Most preferably the generation of intermediate IIIa or IIIb is carried out in tetrahydrofuran.

In one embodiment of the first aspect of the present invention, the conversion of step (ii) further comprises the transformation of intermediate IIIa or of intermediate IIIb into intermediate IV:

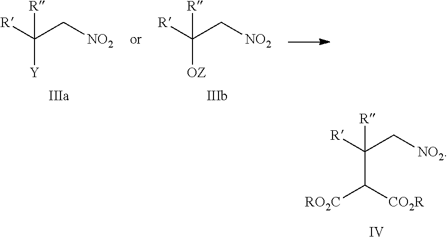

Such a transformation may be achieved for instance by using a carbanion of CH₂(CO₂R)₂. Such a carbanion may be generated using a base, such as a hydride or preferably an alkali metal alkoxide or other alkoxide base, optionally in combination with a metal carbonate such as an alkali metal carbonate. Exemplary alkoxides include for instance MeO⁻, EtO⁻, i-PrO⁻, t-BuO⁻ and PhO⁻. A preferred alkoxide is methoxide, most preferably sodium methoxide. A preferred metal carbonate is sodium carbonate. Preferably the carbanion of CH₂(CO₂R)₂ is generated prior to contact with intermediate IIIa or intermediate IIIb.

The transformation in step (ii) is optionally carried out in an aprotic solvent, preferably an ether solvent or a dipolar aprotic solvent such as N,N-dimethylformamide, dimethyl sulfoxide or acetonitrile. Preferably the transformation is carried out in an ether solvent such as tetrahydrofuran, diisopropyl ether, tert-butyl methyl ether, diethyl ether, or mixtures thereof. Most preferably the transformation is carried out in tetrahydrofuran.

Preferably the transformation is carried out in the same solvent as used for the generation of intermediate IIIa or intermediate IIIb.

Preferably the transformation is achieved without isolating intermediate IIIa or intermediate IIIb.

In one embodiment of the first aspect of the present invention, the conversion of step (iii) of intermediate IV to γ-nitro acid V comprises hydrolysis and decarboxylation.

The hydrolysis and decarboxylation may be achieved for instance using an organic or mineral acid in the presence of water. A preferred mineral acid is hydrochloric acid. Alternatively the hydrolysis and decarboxylation may be achieved using a hydroxide source such as NaOH in the presence of water.

Preferably the hydrolysis and decarboxylation is performed at a temperature greater than 40° C., more preferably greater than 60° C. or greater than 80° C. Most preferably the hydrolysis and decarboxylation is performed at about 100° C.

In another embodiment of the first aspect of the present invention, the reduction of step (iii) of γ-nitro acid V to γ-amino acid VI is performed using catalytic hydrogenation. The catalytic hydrogenation may be performed for instance using a catalyst selected from Pt, Pt/C, PtO₂, Pd, Pd/C, Rh, Ru, Ni or Raney Ni. Preferably the hydrogenation catalyst is selected from Pd/C, Pt/C or PtO₂. Most preferably the hydrogenation catalyst is Pd/C.

The catalytic hydrogenation may be performed for instance in a polar protic solvent such as an alcohol. Preferably the alcohol is selected from methanol, ethanol, 1-propanol, isopropanol, 1-butanol, 2-methyl-1-propanol, t-butanol, 1-pentanol, cyclopentanol, 1-hexanol, cyclohexanol, 1-heptanol or 1-octanol. Most preferably the alcohol is methanol.

Alternatively the reduction of step (iii) of γ-nitro acid V to γ-amino acid VI may be performed using a hydride such as LiAlH₄; Zn, Sn or Fe and an acid; AlH₃—AlCl₃; hydrazine and a catalyst; [Fe₃(CO)₁₂]-methanol; TiCl₃; hot liquid paraffin; formic acid or ammonium formate and a catalyst such as Pd/C; or using sulfides such as NaHS, (NH₄)₂S or polysulfides.

In yet another embodiment of the first aspect of the present invention, where the process comprises step (i) and/or step (ii), step (iii) may instead comprise the hydrolysis, decarboxylation and reduction in any alternate order, such that the overall result of step (iii) is the conversion of intermediate IV into γ-amino acid VI.

In one embodiment of the first aspect of the present invention, the γ-amino acid VI is achiral. For instance the γ-amino acid VI may be gabapentin.

Alternatively the γ-amino acid VI may be a mixture of a chiral γ-amino acid VI, such as a racemic mixture. Preferably the γ-amino acid VI is racemic pregabalin. In such a case the process may further comprise the step of resolving the mixture of the chiral γ-amino acid VI to provide an enantiomerically pure or enantiomerically enriched stereoisomer of the γ-amino acid VI. Preferably the enantiomerically pure or enantiomerically enriched stereoisomer of the γ-amino acid VI is pregabalin. Alternatively, instead of resolving the mixture of the chiral γ-amino acid VI, any of the process intermediates can be resolved, such as intermediate IV or γ-nitro acid V.

In another embodiment of the first aspect of the present invention, the γ-amino acid VI is obtained substantially free of lactam impurity.

A second aspect of the present invention provides a process for the preparation of pregabalin or racemic pregabalin, comprising one or more steps selected from:

(a) reaction of isovaleraldehyde with nitromethane to form 2-hydroxy-4-methyl-1-nitro-pentane; and/or
(b) conversion of 2-hydroxy-4-methyl-1-nitro-pentane to 3-nitromethyl-5-methyl-hexanoic acid; and/or
(c) conversion of 3-nitromethyl-5-methyl-hexanoic acid to pregabalin or racemic pregabalin.

In one embodiment of the second aspect of the present invention, the process comprises two of steps (a) to (c), such as steps (a) and (b), or steps (a) and (c), or steps (b) and (c). Preferably the process comprises all three of steps (a) to (c).

Preferably a carbanion of nitromethane is generated in step (a) with a base, wherein the base is preferably used in a catalytic amount. Optionally the base is not a primary or secondary amine, and preferably is not an amine. Preferably the base is a hydride, an alkoxide or a hydroxide, such as an alkali metal alkoxide or an alkali metal hydroxide. More preferably the base is an alkoxide. Exemplary alkoxides include for instance MeO⁻, EtO⁻, t-BuO⁻ and PhO⁻. A preferred alkoxide is methoxide, most preferably sodium methoxide.

Where a base is used, it is preferably used in 0.001 to 0.040 molar equivalents (eq), more preferably about 0.015 molar equivalents. The preferred quantity of nitromethane with respect to isovaleraldehyde is 1 to 6 molar equivalents, more preferably about 2 molar equivalents.

Step (a) is optionally carried out in an aprotic solvent, preferably an ether solvent or a dipolar aprotic solvent such as N,N-dimethylformamide, dimethyl sulfoxide or acetonitrile.

Preferably step (a) is carried out in an ether solvent, preferably selected from tetrahydrofuran, diisopropyl ether, tert-butyl methyl ether, diethyl ether, or mixtures thereof. Most preferably, the ether solvent is tetrahydrofuran.

Preferably step (b) comprises converting the hydroxy group of 2-hydroxy-4-methyl-1-nitro-pentane to a leaving group and displacing said leaving group with a dialkyl malonate anion, followed by hydrolysis and decarboxylation to afford 3-nitromethyl-5-methyl-hexanoic acid.

Preferably the leaving group is a halo group such as —Cl, —Br or —I, a sulfonate ester group such as a tosylate, brosylate, nosylate, mesylate, tresylate, nonaflate or triflate group, or a carboxylic ester group such as —OCOR^{a} wherein R^{a} is independently selected from hydrogen or an optionally substituted alkyl, alkenyl, alkynyl, aryl, arylalkyl, arylalkenyl or arylalkynyl group. Preferably R^{α} is independently selected from an alkyl, aryl or arylalkyl group optionally substituted with one or more groups selected from —F, —Cl, —Br or —NO₂. More preferably R^{a} is a C₁₋₁₂ alkyl, aryl or arylalkyl group optionally substituted with one or more groups selected from —F, —Cl, —Br or —NO₂, and more preferably R^{a} is a C₁₋₆ alkyl group optionally substituted with one or more groups selected from —F, —Cl or —Br. Most preferably, the leaving group is an optionally substituted acetate group such as a trifluoroacetate group.

Where the leaving group is a carboxylic ester group, it may be generated for instance by the reaction of the hydroxyl group with an acid chloride such as ClCOR^{a}, or an acid anhydride such as R^{a} C(O)OC(O)R^{a}. Preferably acetic anhydride or trifluoroacetic anhydride is used. The acid chloride or acid anhydride may be used for instance in an amount of from 1 to 6 molar equivalents relative to 2-hydroxy-4-methyl-1-nitro-pentane, preferably in an amount of from 1 to 2 molar equivalents, more preferably about 1.3 molar equivalents.

The conversion of the hydroxy group of 2-hydroxy-4-methyl-1-nitro-pentane to a leaving group in step (b) is optionally carried out in an aprotic solvent, preferably an ether solvent or a dipolar aprotic solvent such as N,N-dimethylformamide, dimethyl sulfoxide or acetonitrile. Preferably it is carried out in an ether solvent such as tetrahydrofuran, diisopropyl ether, tert-butyl methyl ether, diethyl ether, or mixtures thereof. Most preferably the conversion of the hydroxy group to a leaving group is carried out in tetrahydrofuran.

Preferably step (b) comprises generating the dialkyl malonate anion with a base, such as a hydride or preferably an alkali metal alkoxide or other alkoxide base, optionally in combination with a metal carbonate such as an alkali metal carbonate. Exemplary alkoxides include for instance MeO⁻, EtO⁻, i-PrO⁻, t-BuO⁻ and PhO⁻. A preferred alkoxide is methoxide. Preferably the alkali metal alkoxide base is sodium methoxide. Preferably the alkali metal carbonate is sodium carbonate. Preferably the dialkyl malonate anion is generated prior to contact with the intermediate formed from the conversion of the hydroxy group of 2-hydroxy-4-methyl-1-nitro-pentane to a leaving group.

Preferably the dialkyl malonate is a di-(C₁₋₁₂ alkyl) malonate, preferably a di-(C₁₋₆ alkyl) malonate. More preferably the dialkyl malonate is a dimethyl, diethyl, dipropyl or dibutyl malonate. Most preferably the dialkyl malonate is dimethyl malonate.

The displacement in step (b) is optionally carried out in an aprotic solvent, preferably an ether solvent or a dipolar aprotic solvent such as N,N-dimethylformamide, dimethyl sulfoxide or acetonitrile. Preferably the displacement is carried out in an ether solvent such as tetrahydrofuran, diisopropyl ether, tert-butyl methyl ether, diethyl ether, or mixtures thereof. Most preferably the displacement is carried out in tetrahydrofuran. Preferably the displacement is carried out in the same solvent as used for the conversion of the hydroxy group of 2-hydroxy-4-methyl-1-nitro-pentane to a leaving group

Preferably the displacement is achieved without isolating the intermediate formed from the conversion of the hydroxy group of 2-hydroxy-4-methyl-1-nitro-pentane to a leaving group.

Preferably step (b) comprises hydrolysis and decarboxylation, for instance using an organic or mineral acid in the presence of water. Most preferably, the mineral acid is hydrochloric acid. Alternatively step (b) may comprise hydrolysis and decarboxylation using a hydroxide source such as NaOH in the presence of water.

Preferably the hydrolysis and decarboxylation is performed at a temperature greater than 40° C., more preferably greater than 60° C. or greater than 80° C. Most preferably the hydrolysis and decarboxylation is performed at about 100° C.

Preferably step (c) comprises catalytic hydrogenation, wherein the hydrogenation catalyst is preferably selected from Pt, Pt/C, PtO₂, Pd, Pd/C, Rh, Ru, Ni or Raney Ni, and is more preferably selected from Pd/C, Pt/C or PtO₂. Most preferably, the hydrogenation catalyst is Pd/C.

The catalytic hydrogenation may be performed for instance in a polar protic solvent such as an alcohol. Preferably the alcohol is selected from methanol, ethanol, 1-propanol, isopropanol, 1-butanol, 2-methyl-1-propanol, t-butanol, 1-pentanol, cyclopentanol, 1-hexanol, cyclohexanol, 1-heptanol or 1-octanol. Most preferably the alcohol is methanol.

Alternatively the reduction of step (c) may be performed using a hydride such as LiAlH₄; Zn, Sn or Fe and an acid; AlH₃—AlCl₃; hydrazine and a catalyst; [Fe₃(CO)₁₂]-methanol; TiCl₃; hot liquid paraffin; formic acid or ammonium formate and a catalyst such as Pd/C; or using sulfides such as NaHS, (NH₄)₂S or polysulfides.

In a process according to the second aspect of the present invention, the racemic pregabalin or pregabalin is preferably obtained substantially free of lactam impurity.

Preferably the process of the second aspect of the present invention further comprises the step of resolving racemic pregabalin to form pregabalin. Alternatively, instead of resolving racemic pregabalin, any of the process intermediates can be resolved, such as 2-carbomethoxy-3-nitromethyl-5-methyl-hexanoic acid methyl ester or 3-nitromethyl-5-methyl-hexanoic acid. Preferably the pregabalin obtained is enantiomerically enriched or enantiomerically pure.

A third aspect of the present invention provides γ-amino acid VI, when prepared by a process according to the first aspect of the present invention. Preferably the γ-amino acid VI is substantially free of lactam impurity. The γ-amino acid VI may be enantiomerically pure or enantiomerically enriched.

A fourth aspect of the present invention provides γ-amino acid VI:

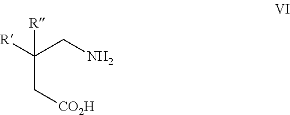

substantially free of lactam impurity, wherein R′ and R″ are as defined above. The γ-amino acid VI may be enantiomerically pure or enantiomerically enriched.

A fifth aspect of the present invention provides racemic pregabalin or enantiomerically enriched pregabalin or enantiomerically pure pregabalin, when prepared by a process according to the first or second aspect of the present invention.

A sixth aspect of the present invention provides racemic pregabalin or enantiomerically enriched pregabalin or enantiomerically pure pregabalin, substantially free of lactam impurity.

Preferably the γ-amino acid according to the third or fourth aspect of the present invention, or the racemic, enantiomerically enriched or enantiomerically pure pregabalin according to the fifth or sixth aspect of the present invention is for treating or preventing epilepsy, pain, neuropathic pain, cerebral ischemia, depression, psychoses, fibromyalgia or anxiety.

A seventh aspect of the present invention provides a pharmaceutical composition comprising the γ-amino acid according to the third or fourth aspect of the present invention, or the racemic, enantiomerically enriched or enantiomerically pure pregabalin according to the fifth or sixth aspect of the present invention. Preferably the pharmaceutical composition is for treating or preventing epilepsy, pain, neuropathic pain, cerebral ischemia, depression, psychoses, fibromyalgia or anxiety.

An eighth aspect of the present invention provides a method of treating or preventing epilepsy, pain, neuropathic pain, cerebral ischemia, depression, psychoses, fibromyalgia or anxiety, the method comprising administering to a patient in need thereof a therapeutically or prophylactically effective amount of the γ-amino acid according to the third or fourth aspect of the present invention, or the racemic, enantiomerically enriched or enantiomerically pure pregabalin according to the fifth or sixth aspect of the present invention, or the pharmaceutical composition according to the seventh aspect of the present invention. The patient is preferably a mammal, most preferably a human.

A ninth aspect of the present invention provides 2-hydroxy-4-methyl-1-nitro-pentane.

A tenth aspect of the present invention provides a compound of formula IVa:

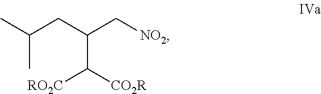

wherein each R is independently an alkyl group. Preferably each R is independently a C₁₋₆ alkyl group, such as methyl, ethyl, propyl or butyl, and most preferably each R is a methyl group, such that the compound of formula IVa is 2-carbomethoxy-3-nitromethyl-5-methyl-hexanoic acid methyl ester.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### DETAILED DESCRIPTION OF THE INVENTION

The present invention provides a simple, convenient and inexpensive method for the preparation of racemic pregabalin (1), which is a key intermediate in the synthesis of pregabalin (2).

The present inventors have observed that the advantages of the present invention are the use of inexpensive, non-hazardous synthetic agents; simple and convenient process conditions; and a very fast synthetic process which has a strict control on the impurity profile of racemic pregabalin (1), which results in obtaining pregabalin (2) of very high chemical and optical purity.

A preferred embodiment of the process of the present invention, illustrated in Scheme 1, comprises four steps: reaction of nitromethane with isovaleraldehyde to form 2-hydroxy-4-methyl-1-nitro-pentane (4); conversion of 2-hydroxy-4-methyl-1-nitro-pentane (4) to 2-carbomethoxy-3-nitromethyl-5-methyl-hexanoic acid methyl ester (5); conversion of 2-carbomethoxy-3-nitromethyl-5-methyl-hexanoic acid methyl ester (5) to 3-nitromethyl-5-methyl-hexanoic acid (6); and conversion of 3-nitromethyl-5-methyl-hexanoic acid (6) to racemic pregabalin (1).

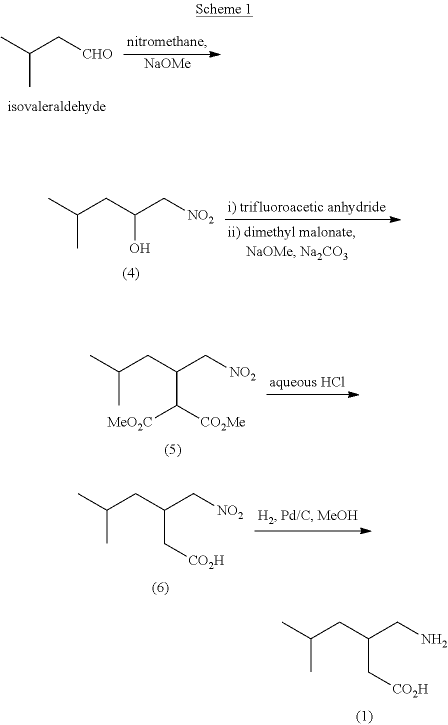

The reagents and solvents illustrated in Scheme 1 are merely illustrative of the present invention and the reactions are not limited by these reagents and solvents. Any suitable alternatives can be used as outlined below.

In the first step, 2-hydroxy-4-methyl-1-nitro-pentane (4) is prepared by a nitro aldol condensation. The process comprises generation of nitromethane carbanion followed by attack of the carbanion on isovaleraldehyde. The nitromethane carbanion can be generated with any suitable base and preferably a catalytic amount of base is used for the generation of the carbanion.

Preferred bases used for the generation of the nitromethane carbanion are alkali metal alkoxides or alkali metal hydroxides, more preferably an alkali metal alkoxide, and most preferably sodium methoxide.

The preferred quantity of base, such as sodium methoxide, chosen for carbanion generation is 0.001 to 0.040 molar equivalents (eq), more preferably about 0.015 molar equivalents.

The nitromethane carbanion is preferably prepared in an organic solvent or a mixture of organic solvents, such as alcoholic, ketonic, hydrocarbon or ether solvents. More preferably, the solvent is an ether, such as tetrahydrofuran (THF), diisopropyl ether, tert-butyl methyl ether, or diethyl ether. The solvent is most preferably tetrahydrofuran.

Preferably the initial carbanion generation is performed at 15-50° C., more preferably at 25-30° C.

The preferred quantity of nitromethane, with respect to the isovaleraldehyde, is 1 to 6 molar equivalents, more preferably around 2 molar equivalents.

In a preferred embodiment, a catalytic amount of sodium methoxide was added to a solution of nitromethane in tetrahydrofuran. After addition of sodium methoxide, the reaction mixture was stirred for 5 minutes to 5 hours, more preferably for about 30 minutes at 25-30° C., and then chilled to about −10 to 15° C., more preferably to about −5 to 0° C. Then, isovaleraldehyde was added with a controlled addition rate, so that the temperature stayed in the range of −5 to 0° C. The reaction mixture was then slowly brought to a preferred temperature of about 25-30° C. and stirred for 6-8 hours. The product was isolated by removal of tetrahydrofuran, preferably under reduced pressure at 35-45° C. The residue was further cooled to 0-10° C. and treated with water to dissolve any inorganic by-products. The product was isolated by extraction with an organic solvent such as ethyl acetate and the solvent removed to obtain 2-hydroxy-4-methyl-1-nitro-pentane (4) as a pale yellow oil.

Preferably, the product (4) is obtained in a yield of 80% or more, preferably 90% or more, preferably 95% or more.

In the second step, the 2-hydroxy-4-methyl-1-nitro-pentane (4) was further converted into 2-carbomethoxy-3-nitromethyl-5-methyl-hexanoic acid methyl ester (5) by transforming the hydroxy group into a suitable leaving group, which may be easily replaced by the anion of dimethyl malonate. Preferably the leaving group is a halo, carboxylate or sulfonate group. When the leaving group is a halo group, it may be a chloro, bromo or iodo group, preferably a bromo group. When the leaving group is a sulfonate group, it may be a mesylate, triflate, tosylate or besylate group. When the leaving group is a carboxylate group, it may be an acetate or a trifluoroacetate group. Most preferably, the leaving group is a trifluoroacetate group.

In a preferred embodiment, the hydroxyl group of 2-hydroxy-4-methyl-1-nitro-pentane (4) is converted to a carboxylate leaving group by reaction with an anhydride reagent such as trifluoroacetic anhydride. The solvent chosen for this reaction is preferably an ether solvent, most preferably tetrahydrofuran.

In a preferred procedure, a solution of 2-hydroxy-4-methyl-1-nitro-pentane (4) was prepared in 0.5 to 10 volumes of tetrahydrofuran, more preferably in about 2 volumes of tetrahydrofuran, and preferably cooled to 0-5° C. Addition of trifluoroacetic anhydride, preferably around 1 to 1.5 molar equivalents, was carried out slowly with controlled rate of addition to avoid an exotherm. In order to avoid impurity formation, addition of trifluoroacetic anhydride was done below 15° C. After the addition was complete, the reaction mixture was stirred preferably for 1 to 10 hours, more preferably for around 1 hour, to allow complete reaction to occur.

Meanwhile, a carbanion solution of a dialkyl malonate, such as dimethyl malonate, can be generated with any suitable base, such as alkali metal alkoxides or hydrides. Sodium methoxide is a preferred base.

In a preferred procedure, a solution of dimethyl malonate carbanion was prepared by using 1 molar equivalent of sodium methoxide in tetrahydrofuran and stirring it for 1-4 hours at 25-30° C. The solution of dimethyl malonate carbanion was added to the trifluoroacetate compound at a controlled rate in such a way that the temperature of the reaction mixture did not increase to more than 10° C. The reaction mixture was stirred for 1 hour at 10° C. and after stirring for 1 hour at 10° C., 1.5 molar equivalents of sodium carbonate at 10° C. were added to increase the basicity of the reaction medium and speed up the reaction. The mixture was heated at 55-60° C. for 4-6 hours to achieve completion of the reaction and the product was isolated by the following aqueous work-up procedures. Tetrahydrofuran was removed under reduced pressure to yield an oily residue and this residue was acidified with 2N HCl to break any inorganic salts. Water was added to the reaction mixture and the product was extracted with ethyl acetate. After removal of the ethyl acetate, 2-carbomethoxy-3-nitromethyl-5-methyl-hexanoic acid methyl ester (5) was isolated as a reddish oil.

Preferably, the product (5) is obtained in a yield of 80% or more, preferably 90% or more, preferably 95% or more.

In the third step, 2-carbomethoxy-3-nitromethyl-5-methyl-hexanoic acid methyl ester (5) is converted to 3-nitromethyl-5-methyl-hexanoic acid (6) in a method preferably comprising the two stages of hydrolysis and decarboxylation. The most preferred reagent for the hydrolysis and decarboxylation is an organic or mineral acid in the presence of water, preferably at a moderately high temperature.

Thus, in a preferred procedure, 2-carbomethoxy-3-nitromethyl-5-methyl-hexanoic acid methyl ester (5) was charged into water and an appropriate ratio of mineral acid, preferably hydrochloric acid, was added. The preferred conditions are 30% aqueous hydrochloric acid and heating at 100-105° C. for 6-8 hours for hydrolysis of the diester product to the diacid and decarboxylation of the diacid to the monoacid to obtain 3-nitromethyl-5-methyl-hexanoic acid (6).

The 3-nitromethyl-5-methyl-hexanoic acid (6) was isolated by extraction with ethyl acetate and the ethyl acetate layer was washed with water to remove any traces of acid from the organic layer. The product was isolated by removal of the ethyl acetate under reduced pressure to obtain 3-nitromethyl-5-methyl-hexanoic acid (6) as a reddish yellow oil.

Preferably, the product (6) is obtained in a yield of 80% or more, preferably 90% or more, preferably 95% or more.

In the fourth step, 3-nitromethyl-5-methyl-hexanoic acid (6) is converted into racemic pregabalin (1) by reduction of the nitro group to an amine group. Aliphatic nitro groups like those in 3-nitromethyl-5-methyl-hexanoic acid (6) can be reduced to amine groups by many reducing agents including catalytic hydrogenation (using hydrogen gas and a catalyst such as Pt, Pt/C, PtO₂, Pd, Pd/C, Rh, Ru, Ni or Raney Ni); Zn, Sn or Fe and an acid; AlH₃—AlCl₃; hydrazine and a catalyst; [Fe₃(CO)₁₂]-methanol; TiCl₃; hot liquid paraffin; formic acid or ammonium formate and a catalyst such as Pd/C; LiAlH₄; and sulfides such as NaHS, (NH₄)₂S or polysulfides. Preferably the reduction is carried out by catalytic hydrogenation using a Pd/C catalyst and hydrogen gas at 25-30° C. at atmospheric pressure.

In a typical procedure, the 3-nitromethyl-5-methyl-hexanoic acid (6) was dissolved in an alcoholic solvent, such as methanol, and the clear solution was further stirred with Pd/C to obtain full mixing of the catalyst. Hydrogen gas was bubbled through the mixture at 25-30° C. for 6-8 hours to achieve complete reduction of the nitro group to an amine group. After completion of the reaction, the catalyst was removed by filtration and the product was isolated by concentration of the solvent under reduced pressure to obtain pregabalin (1) as a pale yellow oil. The pale yellow oil was further converted into solid product by treating it with isopropanol and water. The pregabalin (1) obtained by this method was optionally crystallized, preferably from an isopropanol and water mixture.

Preferably, the product (1) is obtained in a yield of 70% or more, preferably 80% or more, preferably 90% or more, preferably 95% or more. Preferably, racemic pregabalin (1) is obtained substantially free of lactam impurity.

Conversion of racemic pregabalin (1) to pregabalin (2) can be achieved by following well-established and reported routes of resolution, for example, by following the procedure outlined in U.S. Pat. No. 5,637,767. Alternatively, instead of resolving racemic pregabalin (1), any of the process intermediates can be resolved, such as 2-carbomethoxy-3-nitromethyl-5-methyl-hexanoic acid methyl ester (5) or 3-nitromethyl-5-methyl-hexanoic acid (6).

Preferably, the racemic pregabalin (1), the resolved pregabalin (2) and the synthetic intermediates (4), (5) and (6) are obtained on a commercial scale, preferably in batches of 1 kg or more, 10 kg or more, 100 kg or more, 500 kg or more, or 1000 kg or more.

The process of the present invention can be easily adapted for the preparation of γ-amino acids, which are analogous to racemic pregabalin or pregabalin.

The pharmaceutical composition according to the seventh aspect of the present invention can be a solution or suspension form, but is preferably a solid oral dosage form. Preferred dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients in accordance with the invention.

The pharmaceutical composition according to the present invention typically comprises one or more conventional pharmaceutically acceptable excipient(s) such as those selected from the group comprising a filler, a binder, a disintegrant and a lubricant, and optionally further comprises at least one excipient selected from colouring agents, adsorbents, surfactants, film-formers and plasticizers.

As described above, the stable pharmaceutical composition of the invention typically comprises one or more fillers such as microcrystalline cellulose, lactose, sugars, starches modified starches, mannitol, sorbitol and other polyols, dextrin, dextran or maltodextrin; one or more binders such as lactose, starches, modified starch, maize starch, dextrin, dextran, maltodextrin, microcrystalline cellulose, sugars, polyethylene glycols, hydroxypropyl cellulose, hydroxypropyl methyl cellulose, ethyl cellulose, hydroxyethyl cellulose, methyl cellulose, carboxymethyl cellulose, gelatine, acacia gum, tragacanth, polyvinylpyrrolidone or crospovidone; one or more disintegrating agents such as croscarmellose sodium, cross-linked polyvinylpyrrolidone, crospovidone, cross-linked carboxymethyl starch, starches, microcrystalline cellulose or polyacrylin potassium; one or more different glidants or lubricants such as magnesium stearate, calcium stearate, zinc stearate, calcium behenate, sodium stearyl fumarate, talc, magnesium trisilicate, stearic acid, palmitic acid, carnauba wax or silicon dioxide.

If required, the pharmaceutical composition of the present invention may also include surfactants and other conventional excipients. Typical surfactants that may be used are ionic surfactants such as sodium lauryl sulfate or non-ionic surfactants such as different poloxamers (polyoxyethylene and polyoxypropylene copolymers), natural or synthesized lecithins, esters of sorbitan and fatty acids (such as Spano®), esters of polyoxyethylene sorbitan and fatty acids (such as Tween®), polyoxyethylated hydrogenated castor oil (such as Cremophor®), polyoxyethylene stearates (such as Brij®), dimethylpolysiloxane or any combination of the above mentioned surfactants.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

The following paragraphs enumerated consecutively from 1 through 85 provide for various aspects of the present invention. In one embodiment, the present invention provides:

A process for the preparation of a γ-amino acid VI, comprising one or more steps selected from:

(i) the reaction of carbonyl compound I with nitromethane to form alcohol II:

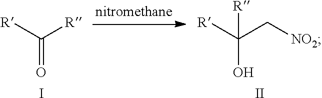

and/or
(ii) the conversion of alcohol II to intermediate IV:

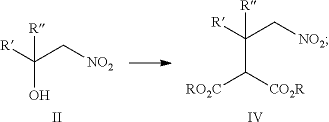

and/or
(iii) the conversion of intermediate IV to γ-nitro acid V, followed by the reduction of γ-nitro acid V to γ-amino acid VI:

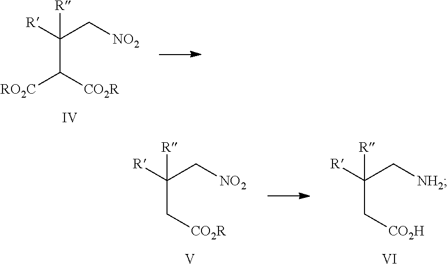

wherein each R is independently an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
wherein R′ and R″ are independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, or both R′ and R″ together with the carbon atom to which they are attached form a cyclic alkyl or cyclic alkenyl group, each of which may optionally be substituted, and each of which may optionally include one or more heteroatoms N, O or S in its carbon skeleton.

A process according to paragraph 1, wherein the process comprises two or three of steps (i) to (iii).

A process according to paragraph 1 or 2, wherein each R is independently an alkyl group.

A process according to paragraph 3, wherein each R is independently a methyl, ethyl, propyl or butyl group.

A process according to paragraph 4, wherein each R is a methyl group.

A process according to any one of the preceding paragraphs, wherein the atoms by which both R′ and R″ are attached to the carbonyl group are either hydrogen or carbon.

A process according to any one of the preceding paragraphs, wherein R′ and R″ are independently hydrogen or an alkyl group, or both R′ and R″ together with the carbon atom to which they are attached form a cyclic alkyl group.

A process according to paragraph 7, wherein R′ and R″ are independently hydrogen or a C₁₋₆ alkyl group, or both R′ and R″ together with the carbon atom to which they are attached form a C₅₋₇ cyclic alkyl group.

A process according to paragraph 8, wherein one of R′ and R″ is hydrogen and the other is i-butyl.

A process according to paragraph 8, wherein both R′ and R″ together with the carbon atom to which they are attached form a cyclohexyl group.

A process according to any one of the preceding paragraphs, wherein a carbanion of nitromethane is generated in step (i) with a base.

A process according to paragraph 11, wherein the base is not an amine.

A process according to paragraph 11 or 12, wherein the base is a hydride, an alkoxide or a hydroxide.

A process according to paragraph 13, wherein the base is sodium methoxide.

A process according to any one of the preceding paragraphs, wherein step (i) is carried out in an ether solvent.

A process according to paragraph 15, wherein the ether solvent is selected from tetrahydrofuran, diisopropyl ether, tert-butyl methyl ether, diethyl ether, or mixtures thereof.

A process according to paragraph 16, wherein the ether solvent is tetrahydrofuran.

A process according to any one of the preceding paragraphs, wherein the conversion of step (ii) comprises the substitution of the hydroxyl group of alcohol II to give intermediate IIIa:

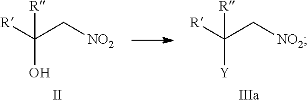

wherein Y is a suitable leaving group.

A process according to paragraph 18, wherein Y is a halo group.

A process according to paragraph 19, wherein Y is —Br.

A process according to any one of paragraphs 1 to 17, wherein the conversion of step (ii) comprises the activation of the hydroxyl group of alcohol II to give intermediate IIIb:

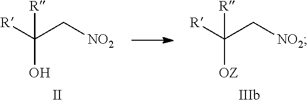

wherein Z is any group capable of enhancing the capacity of a hydroxyl group as a leaving group.

A process according to paragraph 21, wherein Z is selected from a —SO₂R^{α}, —SO₂OR^{α}, —NO₂, —COR^{a}, —P(═O)(OR^{a})₂ or —B(OR^{a})₂ group, wherein each R^{a} is independently selected from hydrogen, a halogen, or an optionally substituted alkyl, alkenyl, alkynyl, aryl, arylalkyl, arylalkenyl or arylalkynyl group, and wherein any two R^{a} groups may together with the atoms to which they are attached form a ring.

A process according to paragraph 22, wherein each R^{a} is independently selected from an alkyl, aryl or arylalkyl group optionally substituted with one or more groups selected from —F, —Cl, —Br or —NO₂.

A process according to paragraph 22 or 23, wherein Z is selected from a —SO₂R^{a}, —SO₂OR^{a} or —COR^{a} group.

A process according to paragraph 24, wherein —OZ is selected from a tosylate, brosylate, nosylate, mesylate, tresylate, nonaflate or triflate group.

A process according to paragraph 24, wherein Z is a —COR^{a} group.

A process according to paragraph 26, wherein Z is an acetyl or trifluoroacetyl group.

A process according to any one of paragraphs 18 to 27, wherein the conversion of step (ii) further comprises the transformation of intermediate IIIa or of intermediate IIIb into intermediate IV:

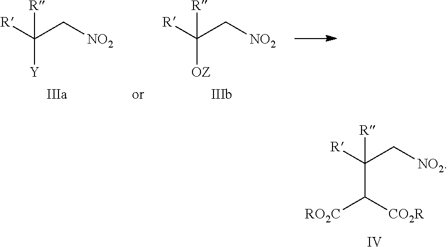

A process according to paragraph 28, wherein the transformation is achieved by using a carbanion of CH₂(CO₂R)₂.

A process according to paragraph 29, wherein the carbanion of CH₂(CO₂R)₂ is generated using an alkoxide base, optionally in combination with a metal carbonate.

A process according to paragraph 30, wherein the alkoxide base is sodium methoxide.

A process according to paragraph 30 or 31, wherein the metal carbonate is sodium carbonate.

A process according to any one of the preceding paragraphs, wherein the conversion of step (iii) of intermediate IV to γ-nitro acid V comprises hydrolysis and decarboxylation.

A process according to paragraph 33, wherein the hydrolysis and decarboxylation is carried out using an organic or mineral acid in the presence of water.

A process according to paragraph 34, wherein the mineral acid is hydrochloric acid.

A process according to any one of the preceding paragraphs, wherein the reduction of step (iii) of γ-nitro acid V to γ-amino acid VI is performed using catalytic hydrogenation.

A process according to paragraph 36, wherein the hydrogenation catalyst is selected from Pd/C, Pt/C or PtO₂.

A process according to paragraph 37, wherein the hydrogenation catalyst is Pd/C.

A process according to any one of the preceding paragraphs, wherein the γ-amino acid VI is achiral.

A process according to paragraph 39, wherein the γ-amino acid VI is gabapentin.

A process according to any one of paragraphs 1 to 38, wherein the γ-amino acid VI is a mixture of a chiral γ-amino acid VI.

A process according to paragraph 41, wherein the γ-amino acid VI is a racemic mixture.

A process according to paragraph 42, wherein the γ-amino acid VI is racemic pregabalin.

A process according to any one of paragraphs 41 to 43, wherein the process further comprises the step of resolving the mixture of the chiral γ-amino acid VI to provide an enantiomerically pure or enantiomerically enriched stereoisomer of the γ-amino acid VI.

A process according to paragraph 44, wherein the enantiomerically pure or enantiomerically enriched stereoisomer of the γ-amino acid VI is pregabalin.

A process according to any one of the preceding paragraphs, wherein the γ-amino acid VI is obtained substantially free of lactam impurity.

A process for the preparation of pregabalin or racemic pregabalin comprising:

(a) reaction of isovaleraldehyde with nitromethane to form 2-hydroxy-4-methyl-1-nitro-pentane;
(b) conversion of 2-hydroxy-4-methyl-1-nitro-pentane to 3-nitromethyl-5-methyl-hexanoic acid; and
(c) conversion of 3-nitromethyl-5-methyl-hexanoic acid to pregabalin or racemic pregabalin.

A process according to paragraph 47, wherein a carbanion of nitromethane is generated in step (a) with a base.

A process according to paragraph 48, wherein the base is used in a catalytic amount.

A process according to paragraph 48 or 49, wherein the base is an alkali metal alkoxide or an alkali metal hydroxide.

A process according to paragraph 50, wherein the base is sodium methoxide.

A process according to any one of paragraphs 47 to 51, wherein step (a) is carried out in an ether solvent.

A process according to paragraph 52, wherein the ether solvent is selected from tetrahydrofuran, diisopropyl ether, tert-butyl methyl ether, diethyl ether, or mixtures thereof.

A process according to paragraph 53, wherein the ether solvent is tetrahydrofuran.

A process according to any one of paragraphs 47 to 54, wherein step (b) comprises converting the hydroxy group of 2-hydroxy-4-methyl-1-nitro-pentane to a leaving group and displacing said leaving group with a dialkyl malonate anion, followed by hydrolysis and decarboxylation to afford 3-nitromethyl-5-methyl-hexanoic acid.

A process according to paragraph 55, wherein the leaving group is a halo group, a sulfonate ester group or a carboxylic ester group.

A process according to paragraph 56, wherein the leaving group is a trifluoroacetate group.

A process according to any one of paragraphs 55 to 57, wherein step (b) comprises generating the dialkyl malonate anion with an alkali metal alkoxide base, optionally in combination with an alkali metal carbonate.

A process according to paragraph 58, wherein the alkali metal alkoxide base is sodium methoxide.

A process according to paragraph 58 or 59, wherein the alkali metal carbonate is sodium carbonate.

A process according to any one of paragraphs 55 to 60, wherein the dialkyl malonate is dimethyl malonate.

A process according to any one of paragraphs 55 to 61, wherein step (b) comprises hydrolysis and decarboxylation using an organic or mineral acid in the presence of water.

A process according to paragraph 62, wherein the mineral acid is hydrochloric acid.

A process according to any one of paragraphs 47 to 63, wherein step (c) comprises catalytic hydrogenation.

A process according to paragraph 64, wherein the hydrogenation catalyst is selected from Pd/C, Pt/C or PtO₂.

A process according to paragraph 65, wherein the hydrogenation catalyst is Pd/C.

A process according to any one of paragraphs 47 to 66, wherein racemic pregabalin or pregabalin is obtained substantially free of lactam impurity.

A process according to any one of paragraphs 47 to 67, wherein the process further comprises the step of resolving racemic pregabalin to form pregabalin.

A process according to any one of paragraphs 47 to 68, wherein enantiomerically enriched or enantiomerically pure pregabalin is obtained.

γ-Amino acid VI, when prepared by a process according to any one of paragraphs 1 to 46.

γ-Amino acid VI:

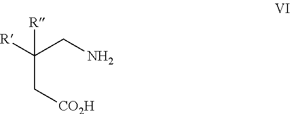

substantially free of lactam impurity, wherein R′ and R″ are as defined in any one of the preceding paragraphs.

A γ-amino acid VI according to paragraph 70 or 71, wherein the γ-amino acid is enantiomerically pure or enantiomerically enriched.

Racemic pregabalin, when prepared by a process according to any one of paragraphs 47 to 67.

Enantiomerically pure or enantiomerically enriched pregabalin, when prepared by a process according to any one of paragraphs 47 to 69.

Racemic pregabalin, substantially free of lactam impurity.

Enantiomerically pure or enantiomerically enriched pregabalin, substantially free of lactam impurity.

A γ-amino acid VI according to paragraph 70, 71 or 72, or racemic pregabalin according to paragraph 73 or 75, or enantiomerically pure or enantiomerically enriched pregabalin according to paragraph 74 or 76, for treating or preventing epilepsy, pain, neuropathic pain, cerebral ischemia, depression, psychoses, fibromyalgia or anxiety.

A pharmaceutical composition comprising a γ-amino acid VI according to paragraph 70, 71, 72 or 77, or racemic pregabalin according to paragraph 73, 75 or 77, or enantiomerically pure or enantiomerically enriched pregabalin according to paragraph 74, 76 or 77.

A pharmaceutical composition according to paragraph 78, for treating or preventing epilepsy, pain, neuropathic pain, cerebral ischemia, depression, psychoses, fibromyalgia or anxiety.

A method of treating or preventing epilepsy, pain, neuropathic pain, cerebral ischemia, depression, psychoses, fibromyalgia or anxiety, the method comprising administering to a patient in need thereof a therapeutically or prophylactically effective amount of a γ-amino acid VI according to paragraph 70, 71, 72 or 77, or racemic pregabalin according to paragraph 73, 75 or 77, or enantiomerically pure or enantiomerically enriched pregabalin according to paragraph 74, 76 or 77, or a pharmaceutical composition according to paragraph 78 or 79.

### 2-Hydroxy-4-methyl-1-nitro-pentane

A compound of formula IVa:

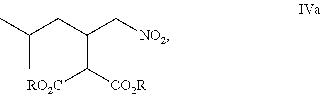

wherein R is independently an alkyl group.

A compound according to paragraph 82, wherein R is independently a methyl, ethyl, propyl or butyl group.

A compound according to paragraph 83, wherein R is a methyl group.

### 2-Carbomethoxy-3-nitromethyl-5-methyl-hexanoic acid methyl ester

Experimental details of a preferred example of the invention are given below.

### Example

### 2-Hydroxy-4-methyl-1-nitro-pentane (4)

A mixture of tetrahydrofuran (1 vol, 1.0 L), nitromethane (2 eq, 1248 ml) and a catalytic amount of sodium methoxide (0.015 eq, 9.4 g) was stirred for 30 minutes to form a slurry of nitromethane anion. The reaction mass was cooled in an ice salt bath at 0° C. and isovaleraldehyde (1 eq, 1 kg) was added, with controlled addition within 1 hour, in such a way that the temperature did not rise above 5° C. After the final addition, the reaction mixture was stirred at 25-30° C. for 6-8 hours. Completion of the reaction was confirmed by TLC.

The tetrahydrofuran was removed under reduced pressure (0.6 kg/cm²) at 50° C. The residue obtained was cooled to 25-30° C. and quenched with water (4 vol, 4.0 L). The product was extracted in ethyl acetate (3 vol, 3.0 L) and separated. The aqueous layer was further extracted with ethyl acetate (2.5 vol, 2.5 L) and the combined organic layers were washed with water (3 vol, 3.0 L). The ethyl acetate was removed under reduced pressure to obtain 2-hydroxy-4-methyl-1-nitro-pentane (4) as a pale yellow oil.

Molar yield: 95-98%

### 2-Carbomethoxy-3-nitromethyl-5-methyl-hexanoic acid methyl ester (5)

Dimethyl malonate (1 eq, 89.76 g), tetrahydrofuran (3 vol, 300 ml) and sodium methoxide (1 eq, 36.7 g) were mixed and the reaction mixture was stirred continuously for 1.5 hours at 25-30° C. to form the enolate of dimethyl malonate.

Simultaneously, 2-hydroxy-4-methyl-1-nitro-pentane (4) (1 eq, 100 g) was dissolved in tetrahydrofuran (2.5 vol, 250 ml) and the clear solution was cooled to 0-5° C. Trifluoroacetic anhydride (1.3 eq, 122.8 ml) was carefully added to the clear solution at 0-5° C. with a controlled rate of addition so that the temperature of the solution did not rise above 15° C.

After the addition of trifluoroacetic anhydride, the reaction mixture was stirred for 1 hour. Completion of the reaction was confirmed by TLC.

After completion of the reaction, the trifluoroacetate derivative was added to the enolate of dimethyl malonate at 10° C. and the mixture was stirred for 1 hour at 10° C. After 1 hour of stirring, sodium carbonate (1.5 eq, 108 g) was added at 10° C. and the reaction mixture was further stirred at 55-60° C. for 6-8 hours. After confirmation of completion of the reaction, the tetrahydrofuran was removed and the reaction mixture was cooled in an ice bath to 10-15° C. At 10-15° C. the residue was acidified with 1N HCl (1 vol, 100 ml) and the product was extracted into ethyl acetate (5 vol, 500 ml). The aqueous layer was further extracted with ethyl acetate (3 vol, 300 ml) and the combined ethyl acetate layers were washed with 5% sodium carbonate solution (5 vol, 500 ml) and water (3 vol, 300 ml). The product, 2-carbomethoxy-3-nitromethyl-5-methyl-hexanoic acid methyl ester (5), was isolated by removal of the ethyl acetate under reduced pressure to obtain a reddish oil.

Molar yield: 85-90%

### 3-Nitromethyl-5-methyl-hexanoic acid (6)

2-Carbomethoxy-3-nitromethyl-5-methyl-hexanoic acid methyl ester (5) (1 eq, 100 g) was charged in water (2 vol, 200 ml) and the mixture was acidified with hydrochloric acid (3.5 vol, 350 ml). The reaction mixture was refluxed at 100-105° C. for 6-8 hours. After completion of the reaction, the mixture was cooled to 25-30° C. and the product was extracted with ethyl acetate (6 vol, 600 ml). The aqueous layer was further extracted with ethyl acetate (3 vol, 300 ml) and the combined ethyl acetate layers were washed with water (2.5 vol, 250 ml). The product, 3-nitromethyl-5-methyl-hexanoic acid (6), was isolated by removal of the ethyl acetate under reduced pressure at 45-50° C.

Molar yield: 85-90%

### Racemic Pregabalin (1)

A mixture of 3-nitromethyl-5-methyl-hexanoic acid (6) (1 eq, 70 g) and methanol (20 vol, 1400 ml) was stirred for 15 minutes to obtain a clear solution. To this clear solution, Pd/C (5%) (20.0 g) was added and the reaction mixture was stirred for a further 15 minutes. Hydrogen gas was bubbled through the mixture at 25-30° C. for 6-8 hours. Completion of the reaction was confirmed by TLC. After completion of the reaction, hydrogen gas bubbling was stopped and the reaction mass was filtered through a Celite® bed. The Celite® bed was further washed with methanol and the methanol was removed completely under reduced pressure at 45-50° C. Isopropanol was charged to the above residual mass and the reaction mass was heated to 60° C. and stirred for 10-20 minutes to obtain a slurry. This slurry was cooled to 25-27° C. and stirred for 2 hours at 25-30° C. The product was filtered and washed with isopropanol (100 ml). The filtered product was dried at 50-55° C. under reduced pressure for 6-8 hours to yield racemic pregabalin (1).

Molar yield: 80%

HPLC purity: 98-99.5%

¹H NMR (D₂O, δ): 0.83 (d, 3H, J=6.48 Hz), 0.87 (d, 3H, J=6.48 Hz), 1.20 (m, 2H), 1.64 (m, 1H), 2.21 (m, 3H), 3.00 (m, 2H).

¹³C NMR (D₂O+DCl+DMSOd₆, δ): 23.39, 23.96, 26.26, 32.92, 39.26, 42.14, 45.02, 179.36.

IR (cm⁻¹, KBr): 2896, 2690, 1645.

The present invention provides an efficient synthesis of racemic pregabalin (1) from isovaleraldehyde in four short steps, which are high yielding and afford a product which is very pure. The conversion of racemic pregabalin (1) to pregabalin (2) can be achieved by following well-established and reported routes of resolution as discussed above.

The difficulties encountered in the prior art for the preparation of racemic pregabalin (1) have been successfully overcome by the process of the present invention and by the use of the novel intermediates.

No trace of the troublesome lactam impurity (3) has been observed by HPLC in the racemic pregabalin (1) or pregabalin (2), when prepared following the process of the present invention.

It will be understood that the present invention has been described above by way of example only. The examples are not intended to limit the scope of the invention. Various modifications and embodiments can be made without departing from the scope and spirit of the invention, which is defined by the following claims only.

1-85. (canceled)

86. A process for the preparation of a γ-amino acid VI, comprising one or more steps selected from:
(i) the reaction of carbonyl compound I with nitromethane to form alcohol II: 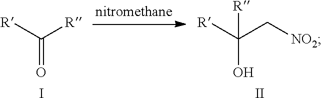
and/or
(ii) the conversion of alcohol II to intermediate IV: 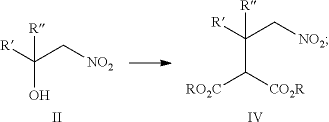
and/or
(iii) the conversion of intermediate IV to γ-nitro acid V, followed by the reduction of γ-nitro acid V to γ-amino acid VI: 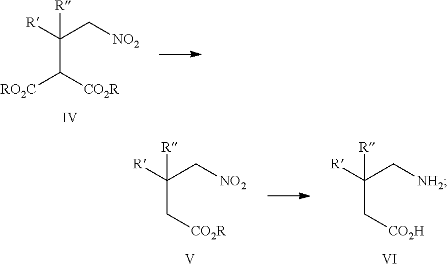
wherein each R is independently an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton; and
wherein R′ and R″ are independently hydrogen or an alkyl, alkenyl, alkynyl, aryl, arylalkyl, arylalkenyl, arylalkynyl, alkylaryl, alkenylaryl or alkynylaryl group, each of which may optionally be substituted, and each of which may optionally include one or more heteroatoms N, O or S in its carbon skeleton, or both R′ and R″ together with the carbon atom to which they are attached form a cyclic alkyl or cyclic alkenyl group, each of which may optionally be substituted, and each of which may optionally include one or more heteroatoms N, O or S in its carbon skeleton.

87. A process according to claim 86, wherein the process comprises two or three of steps (i) to (iii).

88. A process according to claim 86, wherein:
(i) each R is independently an alkyl group; and/or
(ii) each R is independently a methyl, ethyl, propyl or butyl group; and/or
(iii) each R is a methyl group; and/or
(iv) the atoms by which both R′ and R″ are attached to the carbonyl group are either hydrogen or carbon; and/or
(v) R′ and R″ are independently hydrogen or an alkyl group, or both R′ and R″ together with the carbon atom to which they are attached form a cyclic alkyl group; and/or
(vi) R′ and R″ are independently hydrogen or a C₁₋₆ alkyl group, or both R′ and R″ together with the carbon atom to which they are attached form a C₅₋₇ cyclic alkyl group; and/or
(vii) one of R′ and R″ is hydrogen and the other is i-butyl; and/or
(viii) both R′ and R″ together with the carbon atom to which they are attached form a cyclohexyl group.

89. A process according to claim 86, wherein:
(i) a carbanion of nitromethane is generated in step (i) with a base; and/or
(ii) a carbanion of nitromethane is generated in step (i) with a base, wherein the base is not an amine; and/or
(iii) a carbanion of nitromethane is generated in step (i) with a hydride, an alkoxide or a hydroxide; and/or
(iv) a carbanion of nitromethane is generated in step (i) with sodium methoxide; and/or
(v) step (i) is carried out in an ether solvent; and/or
(vi) step (i) is carried out in an ether solvent selected from tetrahydrofuran, diisopropyl ether, tert-butyl methyl ether, diethyl ether, or mixtures thereof; and/or
(vii) step (i) is carried out in tetrahydrofuran.

90. A process according to claim 86, wherein the conversion of step (ii) comprises the substitution of the hydroxyl group of alcohol II to give intermediate IIIa: 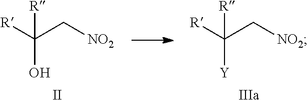
wherein Y is:
(i) a suitable leaving group; and/or
(ii) a halo group; and/or
(iii) —Br.

91. A process according to claim 86, wherein the conversion of step (ii) comprises the activation of the hydroxyl group of alcohol II to give intermediate IIIb: 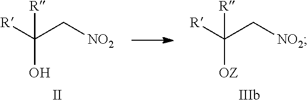
wherein:
(i) Z is any group capable of enhancing the capacity of a hydroxyl group as a leaving group; and/or
(ii) Z is selected from a —SO₂R^{a}, —SO₂OR^{a}, —NO₂, —COR^{a}, —P(═O)(OR^{a})₂ or —B(OR^{a})₂ group, wherein each R^{α} is independently selected from hydrogen, a halogen, or an optionally substituted alkyl, alkenyl, alkynyl, aryl, arylalkyl, arylalkenyl or arylalkynyl group, and wherein any two R^{a} groups may together with the atoms to which they are attached form a ring; and/or
(iii) Z is selected from a —SO₂R^{a}, —SO₂OR^{a}, —NO₂, —COR^{a}, —P(═O)(OR^{a})₂ or —B(OR^{a})₂ group, wherein each R^{a} is independently selected from an alkyl, aryl or arylalkyl group optionally substituted with one or more groups selected from —F, —Cl, —Br or —NO₂; and/or
(iv) Z is selected from a —SO₂R^{a}, —SO₂OR^{a} or —COR^{a} group, wherein each R^{α} is independently selected from hydrogen, a halogen, or an optionally substituted alkyl, alkenyl, alkynyl, aryl, arylalkyl, arylalkenyl or arylalkynyl group, and wherein any two R^{a} groups may together with the atoms to which they are attached form a ring; and/or
(v) Z is selected from a —SO₂R^{a}, —SO₂OR^{a} or —COR^{a} group, wherein each R^{a} is independently selected from an alkyl, aryl or arylalkyl group optionally substituted with one or more groups selected from —F, —Cl, —Br or —NO₂; and/or
(vi) —OZ is selected from a tosylate, brosylate, nosylate, mesylate, tresylate, nonaflate or triflate group; and/or
(vii) Z is a —COR^{a} group, wherein each R^{a} is independently selected from hydrogen, a halogen, or an optionally substituted alkyl, alkenyl, alkynyl, aryl, arylalkyl, arylalkenyl or arylalkynyl group, and wherein any two R^{a} groups may together with the atoms to which they are attached form a ring; and/or
(viii) Z is a —COR^{a} group, wherein each R^{a} is independently selected from an alkyl, aryl or arylalkyl group optionally substituted with one or more groups selected from —F, —Cl, —Br or —NO₂; and/or
(ix) Z is an acetyl or trifluoroacetyl group.

92. A process according to claim 90, wherein the conversion of step (ii) further comprises the transformation of intermediate IIIa or of intermediate IIIb into intermediate IV: 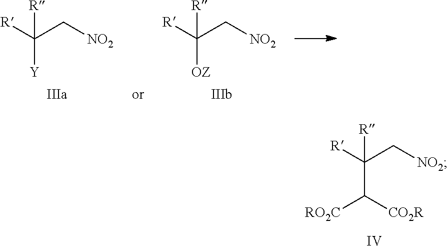
wherein the transformation is achieved by using:
(i) a carbanion of CH₂(CO₂R)₂; and/or
(ii) a carbanion of CH₂(CO₂R)₂, wherein the carbanion of CH₂(CO₂R)₂ is generated using an alkoxide base, optionally in combination with a metal carbonate; and/or
(iii) a carbanion of CH₂(CO₂R)₂, wherein the carbanion of CH₂(CO₂R)₂ is generated using an alkoxide base, optionally in combination with sodium carbonate; and/or
(iv) a carbanion of CH₂(CO₂R)₂, wherein the carbanion of CH₂(CO₂R)₂ is generated using sodium methoxide, optionally in combination with a metal carbonate; and/or
(v) a carbanion of CH₂(CO₂R)₂, wherein the carbanion of CH₂(CO₂R)₂ is generated using sodium methoxide, optionally in combination with sodium carbonate.

93. A process according to claim 91, wherein the conversion of step (ii) further comprises the transformation of intermediate IIIa or of intermediate IIIb into intermediate IV: 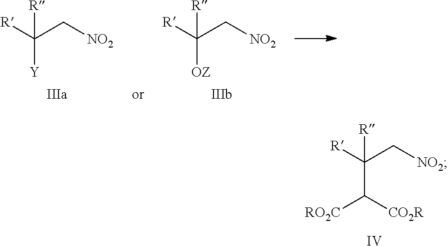
wherein the transformation is achieved by using:
(i) a carbanion of CH₂(CO₂R)₂; and/or
(ii) a carbanion of CH₂(CO₂R)₂, wherein the carbanion of CH₂(CO₂R)₂ is generated using an alkoxide base, optionally in combination with a metal carbonate; and/or
(iii) a carbanion of CH₂(CO₂R)₂, wherein the carbanion of CH₂(CO₂R)₂ is generated using an alkoxide base, optionally in combination with sodium carbonate; and/or
(iv) a carbanion of CH₂(CO₂R)₂, wherein the carbanion of CH₂(CO₂R)₂ is generated using sodium methoxide, optionally in combination with a metal carbonate; and/or
(v) a carbanion of CH₂(CO₂R)₂, wherein the carbanion of CH₂(CO₂R)₂ is generated using sodium methoxide, optionally in combination with sodium carbonate.

94. A process according to claim 86, wherein:
(i) the conversion of step (iii) of intermediate IV to γ-nitro acid V comprises hydrolysis and decarboxylation; and/or
(ii) the conversion of step (iii) of intermediate IV to γ-nitro acid V comprises hydrolysis and decarboxylation, wherein the hydrolysis and decarboxylation is carried out using an organic or mineral acid in the presence of water; and/or
(iii) the conversion of step (iii) of intermediate IV to γ-nitro acid V comprises hydrolysis and decarboxylation, wherein the hydrolysis and decarboxylation is carried out using hydrochloric acid in the presence of water; and/or
(iv) the reduction of step (iii) of γ-nitro acid V to γ-amino acid VI is performed using catalytic hydrogenation; and/or
(v) the reduction of step (iii) of γ-nitro acid V to γ-amino acid VI is performed using catalytic hydrogenation, wherein the hydrogenation catalyst is selected from Pd/C, Pt/C or PtO₂; and/or
(vi) the reduction of step (iii) of γ-nitro acid V to γ-amino acid VI is performed using catalytic hydrogenation, wherein the hydrogenation catalyst is Pd/C.

95. A process according to claim 86, wherein the γ-amino acid VI is:
(i) achiral; and/or
(ii) gabapentin; and/or
(iii) a mixture of a chiral γ-amino acid VI; and/or
(iv) a racemic mixture of a chiral γ-amino acid VI; and/or
(v) racemic pregabalin; and/or
(vi) a mixture of a chiral γ-amino acid VI, and wherein the process further comprises the step of resolving the mixture of the chiral γ-amino acid VI to provide an enantiomerically pure or enantiomerically enriched stereoisomer of the γ-amino acid VI; and/or
(vii) a mixture of a chiral γ-amino acid VI, and wherein the process further comprises the step of resolving the mixture of the chiral γ-amino acid VI to provide enantiomerically pure or enantiomerically enriched pregabalin; and/or
(viii) obtained substantially free of lactam impurity.

96. A process for the preparation of pregabalin or racemic pregabalin comprising:
(a) reaction of isovaleraldehyde with nitromethane to form 2-hydroxy-4-methyl-1-nitro-pentane;
(b) conversion of 2-hydroxy-4-methyl-1-nitro-pentane to 3-nitromethyl-5-methyl-hexanoic acid; and
(c) conversion of 3-nitromethyl-5-methyl-hexanoic acid to pregabalin or racemic pregabalin.

97. A process according to claim 96, wherein:
(i) a carbanion of nitromethane is generated in step (a) with a base; and/or
(ii) a carbanion of nitromethane is generated in step (a) with a base, wherein the base is used in a catalytic amount; and/or
(iii) a carbanion of nitromethane is generated in step (a) with an alkali metal alkoxide or an alkali metal hydroxide; and/or
(iv) a carbanion of nitromethane is generated in step (a) with sodium methoxide; and/or
(v) step (a) is carried out in an ether solvent; and/or
(vi) step (a) is carried out in an ether solvent selected from tetrahydrofuran, diisopropyl ether, tert-butyl methyl ether, diethyl ether, or mixtures thereof; and/or
(vii) step (a) is carried out in tetrahydrofuran.

98. A process according to claim 96, wherein step (b) comprises converting the hydroxy group of 2-hydroxy-4-methyl-1-nitro-pentane to a leaving group and displacing said leaving group with a dialkyl malonate anion, followed by hydrolysis and decarboxylation to afford 3-nitromethyl-5-methyl-hexanoic acid.

99. A process according to claim 98, wherein:
(i) the leaving group is a halo group, a sulfonate ester group or a carboxylic ester group; and/or
(ii) the leaving group is a trifluoroacetate group; and/or
(iii) step (b) comprises generating the dialkyl malonate anion with an alkali metal alkoxide base, optionally in combination with an alkali metal carbonate; and/or
(iv) step (b) comprises generating the dialkyl malonate anion with an alkali metal alkoxide base, optionally in combination with sodium carbonate; and/or
(v) step (b) comprises generating the dialkyl malonate anion with sodium methoxide, optionally in combination with an alkali metal carbonate; and/or
(vi) step (b) comprises generating the dialkyl malonate anion with sodium methoxide, optionally in combination with sodium carbonate; and/or
(vii) the dialkyl malonate is dimethyl malonate; and/or
(viii) step (b) comprises hydrolysis and decarboxylation using an organic or mineral acid in the presence of water; and/or
(ix) step (b) comprises hydrolysis and decarboxylation using hydrochloric acid in the presence of water.

100. A process according to claim 96, wherein step (c) comprises:
(i) catalytic hydrogenation; and/or
(ii) catalytic hydrogenation, wherein the hydrogenation catalyst is selected from Pd/C, Pt/C or PtO₂; and/or
(iii) catalytic hydrogenation, wherein the hydrogenation catalyst is Pd/C.

101. A process according to claim 96, wherein:
(i) racemic pregabalin or pregabalin is obtained substantially free of lactam impurity; and/or
(ii) the process further comprises the step of resolving racemic pregabalin to form pregabalin; and/or
(iii) enantiomerically enriched or enantiomerically pure pregabalin is obtained.

102. γ-Amino acid VI, when prepared by a process according to claim 86.

103. γ-Amino acid VI: 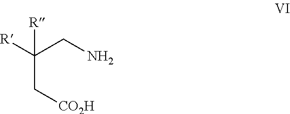
substantially free of lactam impurity, wherein R′ and R″ are independently hydrogen or an alkyl group, or both R′ and R″ together with the carbon atom to which they are attached form a cyclic alkyl group.

104. A γ-amino acid VI according to claim 102, wherein the γ-amino acid is enantiomerically pure or enantiomerically enriched.

105. A γ-amino acid VI according to claim 103, wherein the γ-amino acid is enantiomerically pure or enantiomerically enriched.

106. Racemic pregabalin, enantiomerically pure pregabalin or enantiomerically enriched pregabalin, when prepared by a process according to claim 96.

107. Racemic pregabalin, enantiomerically pure pregabalin or enantiomerically enriched pregabalin, substantially free of lactam impurity.

108. A pharmaceutical composition comprising a γ-amino acid VI according to claim 102.

109. A pharmaceutical composition comprising a γ-amino acid VI according to claim 103.

110. A pharmaceutical composition comprising pregabalin according to claim 106.

111. A pharmaceutical composition comprising pregabalin according to claim 107.

112. A method of treating or preventing epilepsy, pain, neuropathic pain, cerebral ischemia, depression, psychoses, fibromyalgia or anxiety, the method comprising administering to a patient in need thereof a therapeutically or prophylactically effective amount of a γ-amino acid VI according to claim 102.

113. A method of treating or preventing epilepsy, pain, neuropathic pain, cerebral ischemia, depression, psychoses, fibromyalgia or anxiety, the method comprising administering to a patient in need thereof a therapeutically or prophylactically effective amount of a γ-amino acid VI according to claim 103.

114. A method of treating or preventing epilepsy, pain, neuropathic pain, cerebral ischemia, depression, psychoses, fibromyalgia or anxiety, the method comprising administering to a patient in need thereof a therapeutically or prophylactically effective amount of pregabalin according to claim 106.

115. A method of treating or preventing epilepsy, pain, neuropathic pain, cerebral ischemia, depression, psychoses, fibromyalgia or anxiety, the method comprising administering to a patient in need thereof a therapeutically or prophylactically effective amount of pregabalin according to claim 107.

116. 2-Hydroxy-4-methyl-1-nitro-pentane.

117. A compound of formula IVa: 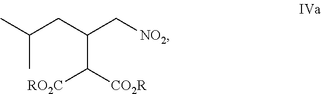
wherein R is independently an alkyl group.

118. A compound according to claim 117, wherein R is:
(i) independently a methyl, ethyl, propyl or butyl group; and/or
(ii) a methyl group.

119. 2-Carbomethoxy-3-nitromethyl-5-methyl-hexanoic acid methyl ester.
